(19) United States
(12) Patent Application Publication
(10) Pub. No.: US 2013/0345427 A1
(43) Pub. Date: Dec. 26, 2013

(54) COLORANT COMPOUND DERIVED FROM GENIPA AMERICANA GENIPIN AND GLYCINE

(71) Applicants: Luis Fernando Echeverry, (US); Juan Fernando Gil, (US); Esteban Vargas, (US)

(72) Inventors: Luis Fernando Echeverry, (US); Juan Fernando Gil, (US); Esteban Vargas, (US)

(21) Appl. No.: 13/532,757

(22) Filed: Jun. 25, 2012

(51) Int. Cl. C07D 401/08

(52) U.S. Cl. 546/112

(57) ABSTRACT

The present invention provides colorant compounds and its molecular structural formulas and methods of isolation of the colorant compounds derived from a reaction of Genipa americana genipin and glycine. The novel compounds were obtained from multiple fractioning by chromatography of the reaction resulting material. The molecular structural formulas resulted from ¹H nuclear magnetic resonance spectroscopy, J-Modulation, H—H Correlation Spectroscopy experiments, and other molecular structural tools analysis.

### BACKGROUND OF THE INVENTION

1. Field of the Invention

The present invention is related to a colorant compound isolated from a reaction of Genipa americana derived genipin and glycine.

2. Description of Prior Art

The blue pigment derived from a reaction of genipin or structural analogs and amino acids have been “found to be an intractable mixture of high molecular polymers on the basis of its chromatographic behavior, un-analyzable 13C-NMR spectrum and by molecular weight measurements” (see Touyama R. et al., Studies on the Blue Pigments Produced from genipin and methylamine. I. Structures of the Brownish-Red Pigments, Intermediates Leading to the Blue Pigments, Chem Pharm. Bull 42, 66, 1994). Therefore, there has been a limited description of the blue pigment material molecular structure since this material is almost soluble only in water due to its very high polarity which results in hard TLC monitoring. A polymer of 9000 molecular weight has been reported (see H. Jnouye, Y. et al., 26th Symposium on the Chemistry of Natural Product, Kyoto, Abstr. pp 577-584, 1983).

The present invention contributes to overcome the lack of knowledge regarding the molecular structures of the blue pigment material derived from a reaction of genipin with an amino-acid.

### SUMMARY OF THE INVENTION

The present invention provides colorant compounds and its molecular structural formulas and methods of isolation of the colorant compounds derived from a reaction of Genipa americana genipin and glycine. The novel compounds were obtained from multiple fractioning by chromatography of the reaction resulting material. The molecular structural formulas resulted from ¹H nuclear magnetic resonance spectroscopy (¹HNMR), J-Modulation (JMOD), H—H Correlation Spectroscopy (COSY ¹H-¹H) experiments, and other molecular structural tools analysis.

Specifically, the present invention provides a colorant compound of the formula 3A (For all purposes in the present Application, formula 3A is for compound No. 3 in the preferred isomeric form):

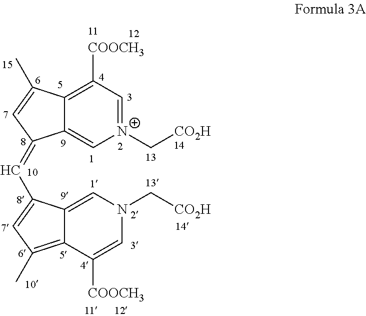 [SMILES: COC(=O)c1c[n+](CC(=O)O)cc2c1C(C)=C/C2=C/c1cc(C)c2c(C(=O)OC)cn(CC(=O)O)cc1-2]

In a less preferred embodiment of the colorant compound of the present invention, said colorant compound, has the isomeric form of formula 3B (For all purposes in the present Application, formula 3B is for compound No. 3 in the a less preferred isomeric form):

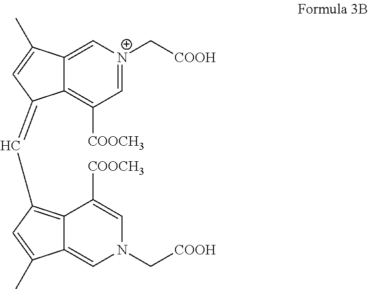 [SMILES: COC(=O)c1c[n+](CC(=O)O)cc2c1/C(=C\c1cc(C)c3cn(CC(=O)O)cc(C(=O)OC)c1-3)C=C2C]

The present invention also provides a method of isolating the colorant compound of formula 3A:

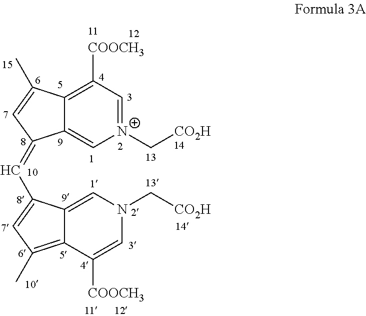 [SMILES: COC(=O)c1c[n+](CC(=O)O)cc2c1C(C)=C/C2=C/c1cc(C)c2c(C(=O)OC)cn(CC(=O)O)cc1-2]

Wherein the methods comprises:
A. Isolating genipin from Genipa Americana juice;
B. Reacting glycine with said genipin to obtain a material soluble in methanol;
C. Separating by chromatography the material soluble in methanol into S1, S2, S3, and S4 fractions.
D. Separating again by chromatography the S3 fraction into S31, S32, S33 and S34 fractions. Isolating by reverse phase chromatography from the S33 fraction the compound of formula I.

In a less preferred embodiment of the method of the present invention, the compound has the isomeric form of Formula 3B:

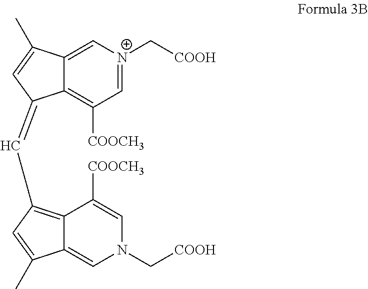 [SMILES: COC(=O)c1c[n+](CC(=O)O)cc2c1/C(=C\c1cc(C)c3cn(CC(=O)O)cc(C(=O)OC)c1-3)C=C2C]

the method comprising:
A. Isolating genipin from Genipa Americana juice;
B. Reacting glycine with said genipin to obtain a material soluble in methanol;
C. Separating by chromatography the material soluble in methanol into S1, S2, S3, and S4 fractions.
D. Separating again by chromatography the S3 fraction into S31, S32, S33 and S34 fractions.
E. Isolating by reverse phase chromatography from the S33 fraction the compound of formula I.

Additional objectives and advantages of the present invention will be more evident in the detailed description of the invention and the claims.

### BRIEF DESCRIPTION OF THE DRAWINGS

FIG. 1. shows chemical formulas for both isomeric forms of compound No. 1.

FIG. 2. shows another representation of the chemical formulas for both isomeric forms of compound No. 1.

FIG. 3. shows chemical formulas for both isomeric forms of compound No. 3.

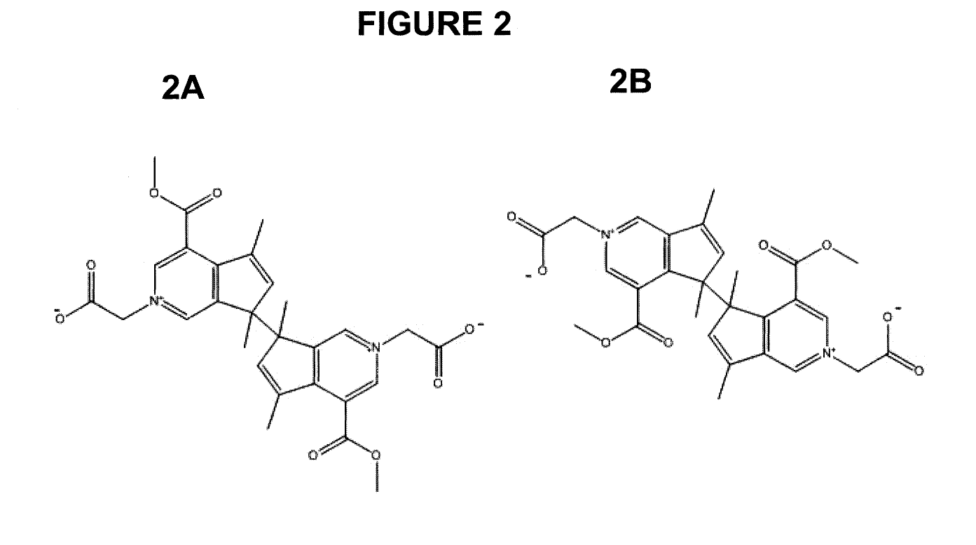

FIG. 2. shows another representation of the chemical formulas for both isomeric forms of compound No. 3.

FIG. 5. shows a nuclear magnetic resonance (NMR) spectroscopy spectra of compound No. 1.

FIG. 6. shows a nuclear magnetic resonance (NMR) spectroscopy spectra of compound No. 3.

FIG. 7. shows the a nuclear magnetic resonance (NMR) for the S31, S32, S33, and S34 fractions derived from the S3 fraction.

### DETAILED DESCRIPTION OF THE INVENTION

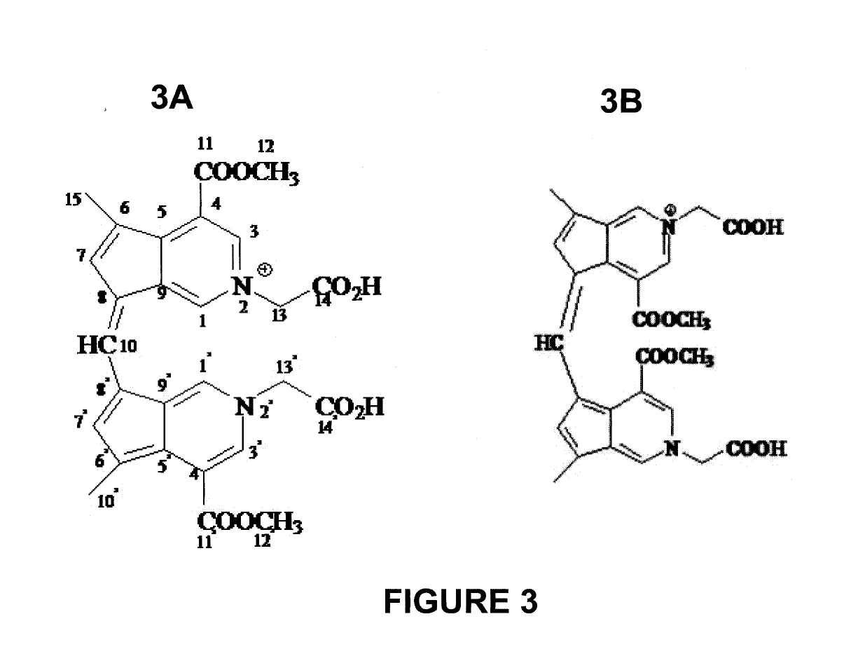

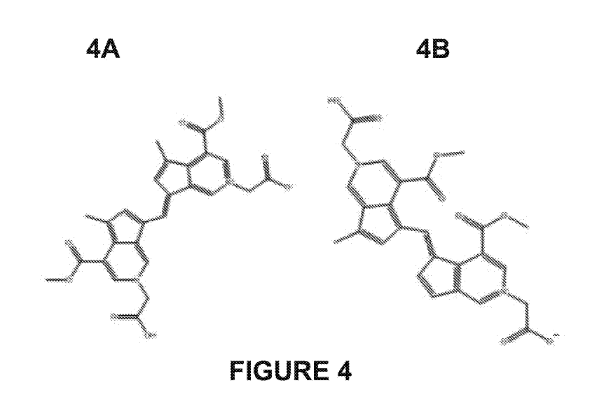

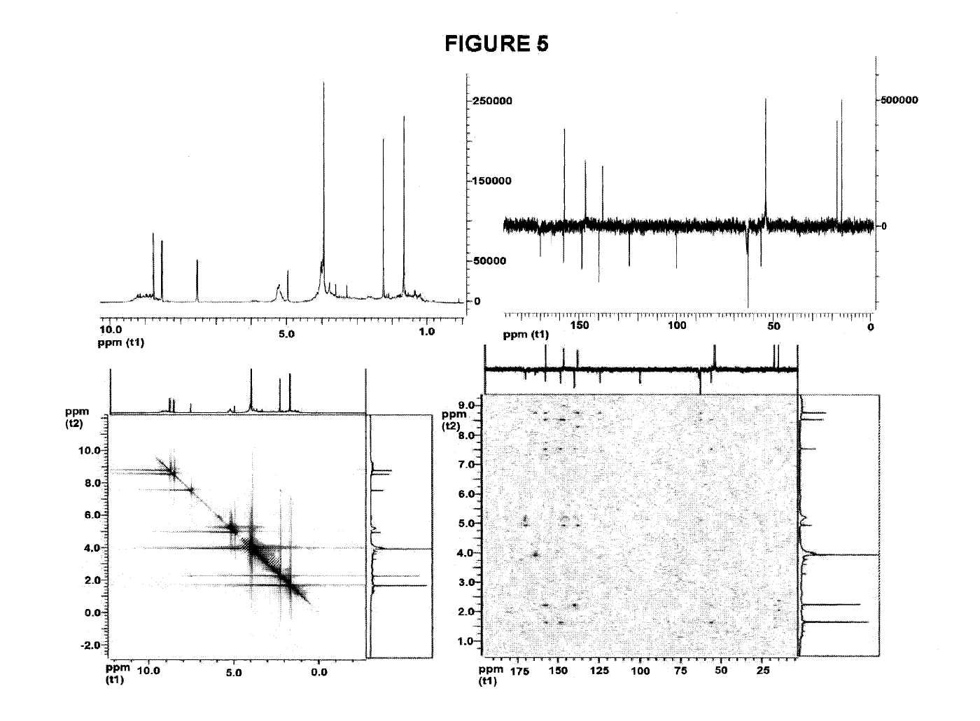

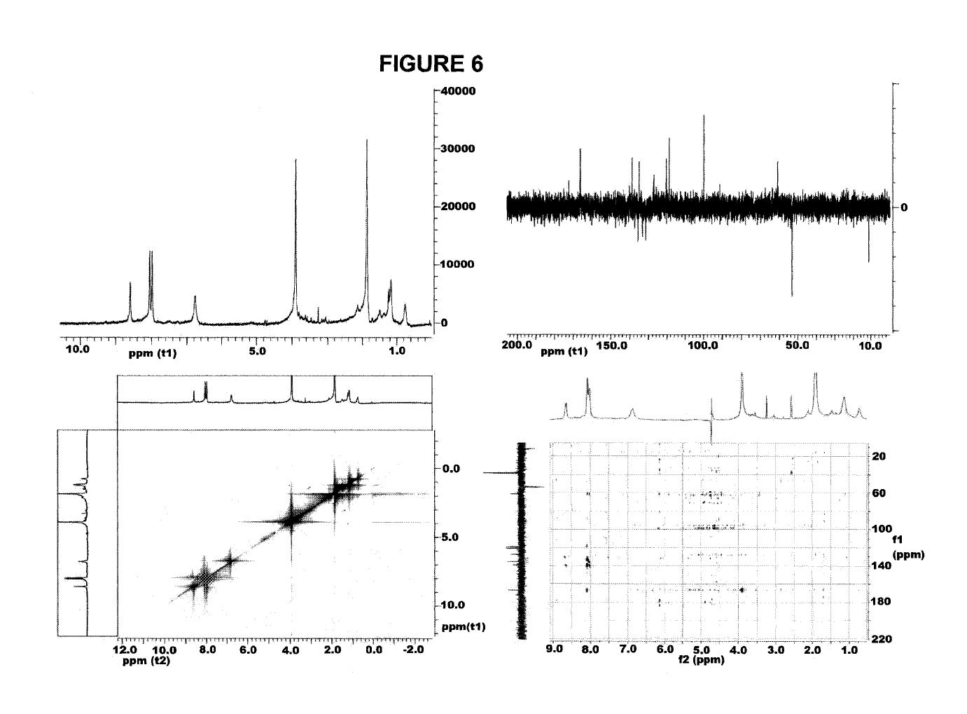

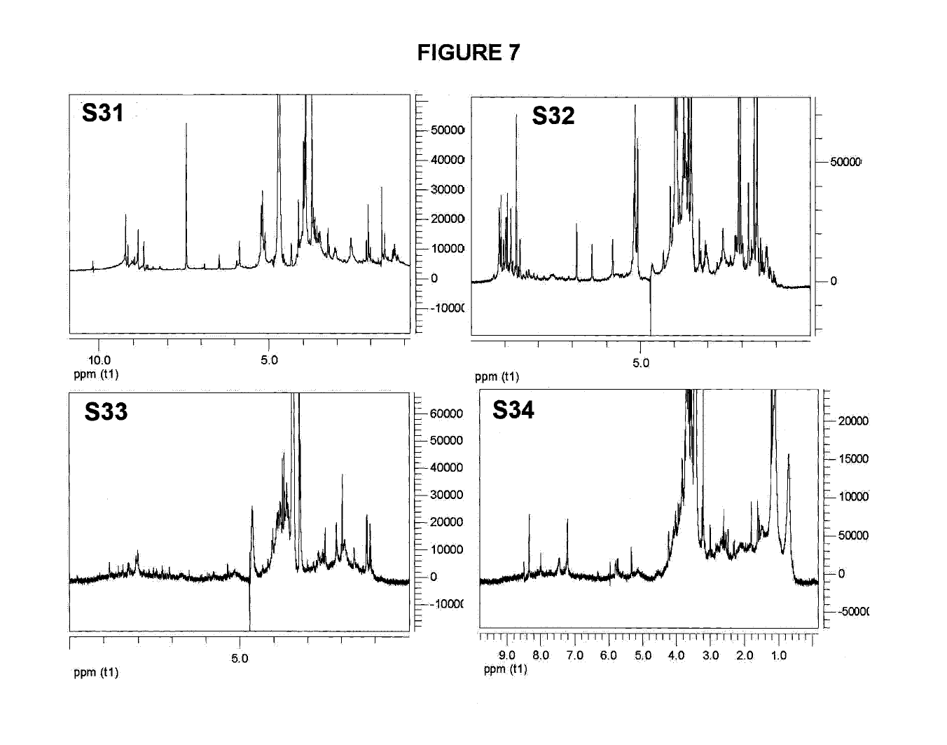

FIGS. 3-3A and 4-4A show representations of the chemical formula for the preferred isomeric form of compound No. 3. Compound No. 3 is a very dark blue colorant substance. FIGS. 3-3B and 4-4B shows the less preferred isomeric form of compound No. 3. FIG. 6 shows the nuclear magnetic resonance (NMR) spectroscopy profile of compound No. 3. Analysis of the NMR spectroscopy profile of compound No. 3. Shows:

¹H NMR (400 MHz, D₂O). δ 8.6, 8.0, 7.9, 6.7, 3.90, 1.8 ppm.

¹³C NMR (100 MHz). δ 172.2, 166.3, 138.8, 135.6, 135.1, 133.3, 131.4, 127.1, 120.46, 118.9, 61.0, 53.3, 11.2 ppm. m/z 505 [M+H]

Further analysis of compound No. 3 showed that:

The mass spectra of the compound 3 displayed m/z=505 [M+H]⁺ in mass spectrometry, so indicating an isomer of the compound previously described. However, the ¹H and ¹³CNM spectra were very different to that one. In the proton spectra, the following singlets were detected: δ 8.0, δ 7.9, and δ 6.7 (2H each one) and one additional singlet at δ 8.6 integrating for 1H. Other signals were a singlet at δ 4.7 (N-CH2) and two methyl groups at δ 3.9 (OCH₃) and δ 1.8 (CH₃ vinyl. According to JMOD experiment, the following carbon atoms were observed too: a carboxyl group at δ 172.2, a methylester at δ 166.3, (COOH), five quaternary carbon atoms at δ 138.8, δ 135.1, δ 127.1, δ 120.4, δ 118.9, four methines at δ 135.6, δ 133.3, δ 131.4, δ 131.4, one methylene (N-—(CH₂) at δ 61.0 and two methyl groups at δ 53.3 (OCH₃) and 11.2 (CH₃ vinyl). The structure of each monomer unit was assigned according to HMBC experiment: signals at δ 7.9 and δ 8.0 were assigned to protons of the pyridil group, since a long range correlation to the N-methylene group at δ 61.0 was detected; additionally the last proton display ³J coupling to the methylester carbonyl at δ 172.2. Besides other important coupling was shown between the singlet at δ 131.4 (C-7) with protons of the methyl group. The low amounts of aromatic and vinyl proton indicated the presence of a symmetric dimeric molecule such as is showed in FIG. 3. Two structures could be assigned to this molecule, according to the relative orientation of the methylester group ((3A and 3B)(FIG. 3), but structure B has a low probability due to steric hindrance, again.

The present invention also provides a method of isolating the colorant compound No. 3.

Wherein the methods comprises:
A. Isolating genipin from Genipa Americana juice;
B. Reacting glycine with said genipin to obtain a material soluble in methanol;
C. Separating by chromatography the material soluble in methanol into S1, S2, S3, and S4 fractions.
D. Separating again by chromatography the S3 fraction into S31, S32, S33 and S34 fractions (FIG. 7). Isolating by reverse phase chromatography from the S33 fraction the compound of formula I.

For the purpose of the present Application the terms S1, S2, S3, S4, and S31, S32, S33 and S34 are a way to define the fractions derived from the described steps of the method. However, these terms (S1, S2, S3, S4, and S31, S32, S33 and S34) cover any fractions obtained by similar chromatographic steps and which could be derived from a reaction genipin and glycine, wherein a S3 similar fraction and S3 derived fractions (of similar NMR spectroscopy as shown in FIG. 7) are produced. FIG. 7 shows the NMR spectroscopy of the S3 fraction derived S31, S32, S33 and S34 fractions.

Although the description presents preferred embodiments of the present invention, additional changes may be made in the form and disposition of the parts without deviating from the ideas and basic principles encompassed by the claims.

### EXAMPLES

Genipin Isolation from Genipa Americana Juice

A solid lyophilized (900 grams) from 10 liters of Genipa americana green juice was Soxhlet extracted with dichloromethane; the generated solvent was evaporated under reduced pressure resulting in a brown residue (240 g); an aliquot of 1 gr was separated by exclusion chromatography by size using, as mobile phase, a mix of hexane/methanol/dichloromethane (2:2:1) from which there were four resulting fractions; genipin was identified in one of the fractions using fine layer chromatography and by comparing with a previously know genipin patter. The fraction containing the genipin was purified multiple times with a chromatographic silica gel column and a hexane/ethyl acetate mobile phase until a pure product (200 mg of genipin) was obtained according to RMN spectra.

### Reaction of Genipin and Glycine

Glycine (200 g) dissolved in water (200 ml) was heated a 70°. Then, genipin (5 g) in methanol (10 ml) was added and the mix was agitated for four hours. The reaction mix was lyophilized and the blue powder was extracted with ethyl-acetate in order to eliminate genipin excess and other low polar components.

### Fractioning of New Components

The blue powder was extracted with methanol (5×100 ml), the generated solvent was evaporated under reduced pressure and a blue resin (2.2gr) was obtained. The blue resin dissolved in methanol 90% was separated in a Sephadex® LH 20 (methanol mobile phase) resulting in four fractions which were denominated (for purposes of this patent Application) S1, S2, S3 and S4.

The S2 fraction was separated using an adsorption resin (Amberlite® XAD-7) using initially 15% ethanol and ending with 95% ethanol. Four sub-fractions were generated from S2. These S2 sub-fractions were denominated (for purposes of this patent Application) M2S1R, M2S2R, M2S3R and M2S4R. The M2S1R was RP-C18 separated several times with different mobile phases (mixes of ethanol-water and methanol-water) until a two compound were obtained, one of those two compounds was denominated compound No. 1 (7 mg). Spectroscopic characteristics of compound No. 1 are:

¹H NMR (400 MHz, D₂O). δ 8.77, 8.53, 7.54, 5.30-4.95, 3.94, 2.25, 1.66 ppm.

¹³C NMR (100 MHz). δ 170.0, 164.16, 157.80, 157.44, 148.29, 146.41, 139.76, 137.83, 124.16, 63.35, 62.6, 56.19, 53.89, 17.43, 14.93 ppm.

Further analysis of compound No. 1 showed that:

In ¹H NMR displayed a few signals: two aromatic protons as singlets at δ 8.77 and 8.53, a vinylic proton at 7.54, a singlet at 4.95, (2H) and three singlets integrating for 3H each one at 3.94 (OCH₃), 2.25 (vynilic methyl group), and 1.66.

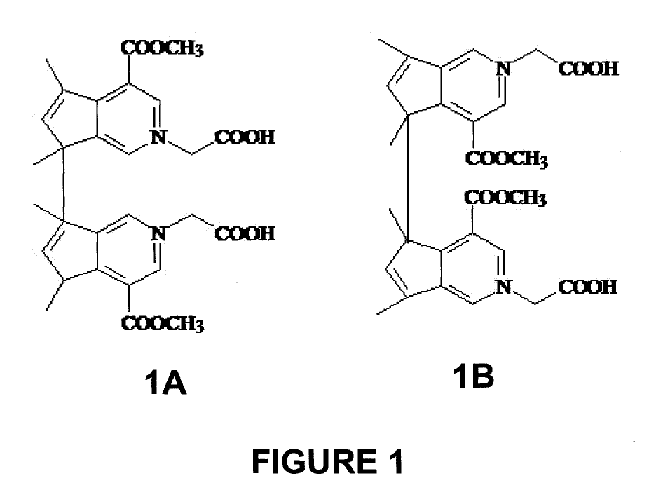

The JMOD experiment displayed the following signals: three methyl groups at 14.93, 17.43 and 53.89, one methylene at 62.68, assignable to a methylene derived from glycine, three methine at 157.44, 146.41, 137.83 and finally, seven quaternary carbon atoms at 170.00 (carboxylic), 164.16 (methyl ester carbonyl), 157.80, 148.29, 139.76, 124.16 and 53.89. So, the genipin moiety and glycine residue has been conserved, but molecule now is aromatic with a pyridil residue, due to position of the protons and carbons atoms in NMR spectra. However, a new methyl group been appeared in the structure and his position was assignable on the basis of JMOD, HMQC and HMBC experiments. So, COSY 1H-1H showed an allylic connectivity between methyl group at 2.25 with vynilic proton at 7.54; in the HMBC experiment this proton displayed 3J coupling to these methyl (157.44 in 13C NMR) and the aliphatic methyl group at 14.93 (1.66 in 1H NMR), which in turn, establish a correlation to the quaternary carbon atom at 53.89 and aromatic at 157.80 and 148.29. Other long range connectivities detected were: N-CH2 (62.68) to both aromatic protons at 8.77 and 8.53, and the former to methylester carbonyl. Finally, MS exhibited a m/z 522 [W+H] indicating a symmetric dimeric molecule, as can be seen in FIGS. 1 and 2. The connecting bridge between monomers was deduced through C-8 and C-8′ carbon atoms, since apparition of a methyl group as a singlet, which is mutually coupled to the other methyl group in the HMBC experiment. There are two possible isomers as it is shown in 1A, 1B, 2A, 2B of FIGS. 1 and 2.

The S3 fraction was separated by chromatography with Sephadex® using a 95% methanol mobile phase generating four S3 fractions that for the purpose of this patent Application were denominated S31, S32, S33, and S34. The S33 fraction was separated several times by RP-C18 reverse chromatography using different mobile phases (mixes of ethanol-water and methanol-water) until a compound, which was denominated compound No. 3 (4 mg) was obtained. The Spectroscopic characteristics of compound No. 3 are:

¹H NMR (400 MHz, D₂O). δ 8.6, 8.0, 7.9, 6.7, 3.90, 1.8 ppm.

¹³C NMR (100 MHz). δ 172.2, 166.3, 138.8, 135.6, 135.1, 133.3, 131.4, 127.1, 120.46, 118.9, 61.0, 53.3, 11.2 ppm. m/z 505 [M+H]

Further analysis of compound No. 3 showed that:

The mass spectra of the compound 3 displayed m/z=505 [M+H]⁺ in mass spectrometry, so indicating an isomer of the compound previously described. However, the ¹H and ¹³CNM spectra were very different to that one. In the proton spectra, the following singlets were detected: δ 8.0, δ 7.9, and δ 6.7 (2H each one) and one additional singlet at δ 8.6 integrating for 1H. Other signals were a singlet at δ 4.7 (N-CH2) and two methyl groups at δ 3.9 (OCH₃) and δ 1.8 (CH₃ vinyl. According to JMOD experiment, the following carbon atoms were observed too: a carboxyl group at δ 172.2, a methylester at δ 166.3, (COOH), five quaternary carbon atoms at δ 138.8, δ 135.1, δ 127.1, δ 120.4, δ 118.9, four methines at δ 135.6, δ 133.3, δ 131.4, δ 131.4, one methylene (N-—(CH₂) at δ 61.0 and two methyl groups at δ 53.3 (OCH₃) and 11.2 (CH₃ vinyl). The structure of each monomer unit was assigned according to HMBC experiment: signals at δ 7.9 and δ 8.0 were assigned to protons of the pyridil group, since a long range correlation to the N-methylene group at δ 61.0 was detected; additionally the last proton display ³J coupling to the methylester carbonyl at δ 172.2. Besides other important coupling was shown between the singlet at δ 131.4 (C-7) with protons of the methyl group. The low amounts of aromatic and vinyl proton indicated the presence of a symmetric dimeric molecule such as is showed in FIG. 3. Two structures could be assigned to this molecule, according to the relative orientation of the methylester group ((3A and 3B)(FIG. 3), but structure B has a low probability due to steric hindrance.

1. A colorant compound of the formula 3A:

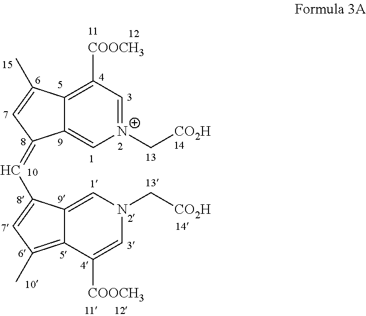 [SMILES: COC(=O)c1c[n+](CC(=O)O)cc2c1C(C)=C/C2=C/c1cc(C)c2c(C(=O)OC)cn(CC(=O)O)cc1-2]

2. The colorant compound of claim, wherein the compound has the isomeric form of formula 3B:

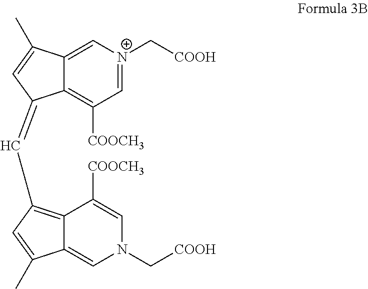 [SMILES: COC(=O)c1c[n+](CC(=O)O)cc2c1/C(=C\c1cc(C)c3cn(CC(=O)O)cc(C(=O)OC)c1-3)C=C2C]

3. A method of isolating the compound of formula 3A: 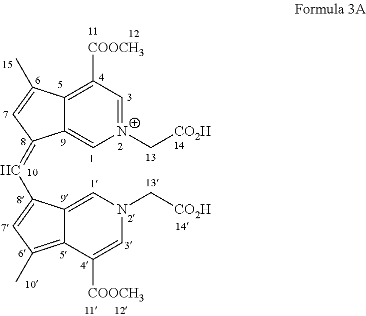 [SMILES: COC(=O)c1c[n+](CC(=O)O)cc2c1C(C)=C/C2=C/c1cc(C)c2c(C(=O)OC)cn(CC(=O)O)cc1-2]
the method comprising:
A. Isolating genipin from Genipa Americana juice;
B. Reacting glycine with said genipin to obtain a material soluble in methanol;
C. Separating by chromatography the material soluble in methanol into S1, S2, S3, and S4 fractions.
D. Separating again by chromatography the S3 fraction into S31, S32, S33 and S34 fractions.
E. Isolating by reverse phase chromatography from the S33 fraction the compound of formula I.

4. A method of claim 3, wherein the compound has the isomeric form of Formula 3B: 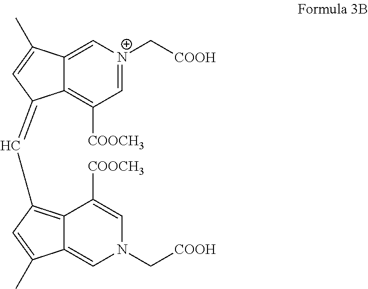 [SMILES: COC(=O)c1c[n+](CC(=O)O)cc2c1/C(=C\c1cc(C)c3cn(CC(=O)O)cc(C(=O)OC)c1-3)C=C2C]
the method comprising:
A. Isolating genipin from Genipa Americana juice;
B. Reacting glycine with said genipin to obtain a material soluble in methanol;
C. Separating by chromatography the material soluble in methanol into S1, S2, S3, and S4 fractions.
D. Separating again by chromatography the S3 fraction into S31, S32, S33 and S34 fractions.
E. Isolating by reverse phase chromatography from the S33 fraction the compound of formula I.
